(19) United States
(12) Patent Application Publication
(10) Pub. No.: US 2008/0200696 A1
(43) Pub. Date: Aug. 21, 2008

(54) SEPARATION PROCESS USING AROMATIC-SELECTIVE POLYMERIC MEMBRANES

(76) Inventors: Jeffrey T. Miller, Naperville, IL (US); Bo Chen, Aurora, IL (US); Craig W. Colling, Warrenville, IL (US); George A. Huff, Naperville, IL (US); Brian Henley, Sandwich, IL (US); William John Koros, Atlanta, GA (US); Raymond W. Chafin, Pearland, TX (US)

Correspondence Address: CAROL WILSON;BP AMERICA INC., MAIL CODE 5 EAST, 4101 WINFIELD ROAD, WARRENVILLE, IL, 60555, US

(21) Appl. No.: 11/428,851

(22) Filed: Jul. 6, 2006

(51) Int. Cl. C07D 209/38

(52) U.S. Cl. 548/485

(57) ABSTRACT

Processes are disclosed for production of value-added products from fluid admixtures of hydrocarbon compounds at least one of which is an aromatic hydrocarbon compound, by means of one or more devices using perm-selective polymeric membranes. More particularly, processes of the invention comprise separations using aromatic-selective polymeric materials comprising long-chain polymeric molecules in which recurring amide and imide linkages are part of the main polymer chain. Processes of the invention advantageously employ aromatic-selective membranes to separate an aromatic enriched stream from gaseous and/or liquid mixtures comprising one or more aromatic hydrocarbon compounds thereby producing a stream comprising the remaining compounds which may include alkenes and/or alkanes containing 3 or more carbon atoms, and/or alicyclic hydrocarbons. Processes of the invention are particularly useful for recovery of meta-xylene and para-xylene products from liquid mixtures even containing ethylbenzene as well as the three xylene isomers.

### TECHNICAL FIELD

The present invention relates to processes for recovery of value added products from a fluid admixture of hydrocarbon compounds least one of which is an aromatic hydrocarbon compound, by means of one or more devices using perm-selective polymeric membranes. More particularly, processes of the invention comprise separations using aromatic-selective polymeric membranes comprising long-chain polymeric molecules in which recurring amide and imide linkages are part of the main polymer chain, i.e., poly(amide)imides. Processes of the claimed invention advantageously employ aromatic-selective membranes to separate an aromatic enriched stream from gaseous and/or liquid mixtures comprising one or more aromatic hydrocarbon compound thereby producing a stream comprising the remaining compounds which may include alkenes and/or alkanes containing 3 or more carbon atoms, and/or alicyclic hydrocarbons. Processes of the invention are particularly useful for recovery of meta-xylene and para-xylene products from liquid mixtures, even mixtures containing ethylbenzene as well as one or more isomer of xylene, i.e., ortho-xylene, meta-xylene, and para-xylene.

### BACKGROUND OF THE INVENTION

Although in some instances it is possible to separate gas and liquid mixtures to some extent by selective permeation through thin film membranes of various compositions and structure, there exists a strong need for processes that selectively recover an aromatic hydrocarbon compound from a fluid admixture of other petroleum derived compounds. For example, gas separation through polymer membranes has been commercially applied for the separation of H₂ from NH₃, methanol and refinery streams. CO₂ from natural gas and N₂ from air since the later 1970's. Pervaporation is the selective permeation of liquid mixtures with subsequent evaporation and has been commercially applied for drying of ethanol.

In the pervaporation process, one or more of the liquid components preferentially adsorb on one side of a dense polymer membrane, diffuse through and desorb into the gas phase at the opposite membrane surface. Attractive membranes are those with physical stability under conditions of operation and which exhibit high flux and acceptable separation factors. Identification of a suitable membrane is the most important hurdle to development of an economic separation process. The separation selectivity is generally thought to be controlled by one of two properties, differences in the selective solubility of the permeate into the polymer, or differences in size of the diffusing molecules through the pores of the membrane (See E. K. Lee and W. J. Koros, Encyclopedia of Physical Science and Technologies, 3rd Ed., Academic Press, 2002, pp 279-344).

The C₈ aromatics exist as ethylbenzene and three isomers of xylene (dimethylbenzenes) which are separated only with great difficulty because they have boiling points which are very close together. While the demand for para-xylene remains high, demand for meta-xylene is steadily increasing. Meta-xylene is used for the manufacture of insecticides, isophthalic acid or alkyd resins. Para-xylene is used in the manufacture of terephthalic acid which in turn is subsequently employed in the manufacture of various synthetic fibers, such as polyester. Ortho-xylene can be used as material for plasticizers. Benzene di- and tri-carboxylic acids have wide industrial application including the manufacture of polyesters, polyamides, fibers and films. For commercial manufacture of these products the required source of high purity benzene di- and tri-carboxylic acids may be obtained from a corresponding substituted aromatic compound by catalytic oxidation of methyl moieties to carboxylic acid moieties, advantageously in a liquid-phase medium.

While polymer membranes of many compositions have been extensively studied for separation of gases, less has been done for the separation of organic mixtures, especially C₈ aromatic-containing hydrocarbon mixtures.

U.S. Pat. Appl.: 20050167338 and 20050171395 disclose integrated processes that comprise separations by means of one or more devices using polymeric membranes coupled with recovery of purified products by means of fractional crystallization and/or selective sorption. These processes are said to be particularly useful for recovery of a very pure aromatic isomer when processing aromatic starting materials, for example, a pure para-xylene product from liquid mixtures even containing ethylbenzene as well as the three xylene isomers.

Polyethylene films treated under different conditions show some selective permeation of para-xylene over ortho-xylene. Cellulose acetate modified by dinitrophenylchloride groups was also reported to demonstrate selective permeation of para-xylene over ortho-xylene. Due to their physical and chemical properties, these polymers do not have the stability or exhibit the desired selective separations at operating conditions needed for separating organic mixtures containing aromatic hydrocarbons.

Recent publications have described attempts to separate C₈ aromatics, ethylbenzene and xylenes isomers with several different commercial polymers, but with very limited results. Some commercial polymers have demonstrated low separation factors of para-xylene over ortho-xylene, and limited permeation flux. Cross-linked polyimides also show higher permeation of para-xylene over ortho-xylene. (See M. Schleiffelder, C. Staudt-Bickel, Reactive and Fundamental Polymers, 49, 2001, pp 205-213).

With these examples demonstrate that polymer membranes have been prepared and tested for separation of xylene isomers by pervaporation, none of these papers, however, describes polymer materials with selectivities greater than 1.8 for separation of xylene isomers.

There is a need for a cost effective method of producing value-added products from fluid admixtures of hydrocarbon compounds at least one of which is an aromatic hydrocarbon compound, by means of one or more devices using perm-selective polymeric membranes.

Accordingly, it is an object of the invention to overcome one or more of the problems described above.

There is a need for a cost effective method of producing high purity para-xylene and/or meta-xylene from a C₈ aromatic mixture containing para-xylene, meta-xylene, ortho-xylene and ethylbenzene.

Beneficially, new and improved processes should comprise separations using aromatic-selective polymeric membrane materials that have more than one of the following properties/advantages: a) excellent selectivity and permeability, b) sustained selectivity over extended periods, c) physical and chemical stability under conditions of operation, and d) very large useable surface area by use of hollow-fiber and/or spiral wound membranes.

Advantageously, processes of the invention also provide for simultaneous recovery of para-xylene having an improved purity from liquid mixtures of aromatic compounds, even containing ethylbenzene as well as the three xylene isomers.

### SUMMARY OF THE INVENTION

In broad aspect, the present invention is directed to processes for production of value-added products from fluid admixtures of hydrocarbon compounds by means of one or more devices using perm-selective polymeric membranes. Typically, at least one of the products is an aromatic hydrocarbon compound. More particularly, processes of the invention comprise separations using aromatic-selective membranes comprising long-chain polymeric molecules in which recurring amide and imide linkages are part of the main polymer chain. Processes of the invention advantageously employ aromatic-selective membranes to separate an aromatic enriched stream from mixtures comprising one or more aromatic hydrocarbon compounds thereby producing a stream comprising the remaining compounds, which may include alkenes and/or alkanes containing 3 or more carbon atoms, and/or alicyclic hydrocarbons. Processes of the invention are particularly useful for recovery of meta-xylene and para-xylene products from fluid mixtures even containing ethylbenzene as well as the three xylene isomers.

In one aspect, the invention provides a process for recovery of one or more aromatic hydrocarbon compounds in admixture with other organic compounds which process comprises: (a) contacting a fluid mixture comprising two or more aromatic hydrocarbon compounds, with a first side of a perm-selective membrane comprising long-chain polymeric molecules in which recurring amide and imide linkages are part of the main polymer chain; and (b) selectively permeating at least one aromatic hydrocarbon compound of the mixture through the membrane to a permeate side that is opposite the first side, thereby exhibiting a separation factor of one aromatic hydrocarbon over another compound in a range upward from about 1.5. For recovery of some desired products, it is advantageous that the polymeric material is annealed at elevated temperatures in a range upward from about the glass transition temperature of the polymeric membrane material.

Useful perm-selective membranes of the invention comprise a polymer derived from the reaction product of a carbocyclic aromatic primary diamine and an acyl halide derivative of trimellitic anhydride which contains at least one acyl halide group and that in the 4-ring position.

Another class of useful perm-selective membranes comprise a polymer derived from the reaction product of an aromatic diisocyanate and a tricarboxylic acid anhydride derived from reactants selected from the group of reactant pairs consisting of (a) trimellitic anhydride and toluene diisocyanate and (b) trimellitic anhydride chloride and toluene diamine. More particularly, perm-selective membrane of the invention comprises a polymer. Beneficially, membranes of this invention comprises a polymer derived from the reaction product of trimellitic anhydride chloride and p-methylenediamine (MDA). Particularly useful membranes of the invention comprises a polymer derived from the reaction product of trimellitic anhydride chloride and a mixture of 4,4′-oxydianiline (ODA) and m-phenylenediamine (m-PDA) by a condensation polymerization.

This invention contemplates the treatment of a fluid feedstock, e.g. various type organic materials, especially a fluid mixture of compounds of petroleum origin. In general, the fluid feedstock is a liquid mixture comprising a more selectively permeable component and a less permeable component.

Particularly useful embodiments of the invention provide processes for recovery of one or more aromatic hydrocarbon compounds from fluid mixtures, that comprise para-xylene and least one other C₈ aromatic compound. For example, fluid mixtures comprise para-xylene and at least one other isomer of xylene, ethylbenzene or mixtures thereof, typically an equilibrium mixture of the xylene isomers. Where a fluid mixture comprises the three isomers of xylene and optionally ethylbenzene, the permeation advantageously exhibits a separation factor for para-xylene/meta-para-xylene (pX/mX) of at least 1.5.

Other suitable fluid admixtures comprise at least one aromatic hydrocarbon compound having 8 or more carbon atoms, and the compounds containing 4 or more carbon atoms that are selected from the group consisting of alkenes, alkanes and alicyclic hydrocarbons.

Processes of the invention beneficially utilizes a plurality of hollow fiber and/or spiral wound perm-selective membranes which under a suitable differential of a driving force exhibit a permeability of at least 0.1 Barrer for at least one of the isomers of xylene or ethylbenzene.

The selective permeation may be carried out at any suitable operating conditions, for example at temperatures in a range downward from about 220° C. to about 70° C. and feed pressures up to 900 psia. Advantageously the permeation exhibits a para-xylene permeability of at least 0.1 Barrer.

In another aspect, the invention provides a process for recovery of one or more aromatic hydrocarbon compounds in admixture with other organic compounds which process comprises: (a) contacting a fluid admixture comprising hydrocarbon compounds having 4 or more carbon atoms, that includes at least one aromatic hydrocarbon compound, with a first side of a perm-selective membrane comprising long-chain polymeric molecules in which recurring amide and imide linkages are part of the main polymer chain, and the poly(amide)imide membrane material exhibited a Stability Rating of a level 3 pass (defined herein below); and (b) selectively permeating at least one aromatic hydrocarbon compound of the mixture through the membrane to a permeate side that is opposite the first side, thereby exhibiting a separation factor of one aromatic hydrocarbon over another compound in a range upward from about 2.5.

In yet another aspect, the invention provides a process for recovery of one or more aromatic hydrocarbon compounds in admixture with other organic compounds which process comprises: (a) contacting a fluid admixture comprising two or more hydrocarbon compounds each having at least 5 carbon atoms, that includes at least one aromatic hydrocarbon compound, with a first side of a hollow fiber membrane comprising long-chain polymeric molecules in which recurring amide and imide linkages are part of the main polymer chain; and (b) selectively permeating at least one aromatic hydrocarbon compound of the mixture through the membrane to a permeate side that is opposite the first side, thereby exhibiting a separation factor of one aromatic hydrocarbon over another compound in a range upward from about 5, 10 or higher for best results. The more useful poly(amide)imide membrane materials of the invention exhibited a Stability Rating above a level 1 pass, advantageously a Stability Rating of a level 3 pass.

In recovery processes of the invention using hollow fiber and/or spiral wound membranes, the selective permeation is carried out under suitable operating conditions whereby membranes exhibit a para-xylene permeability in a range upward from 0.1 Barrer, and at least 0.5 Barrer for best results. Suitable operating conditions for selective permeation using these membranes beneficially include temperatures in a range downward from about 220° C. to about 70° C. and feed pressures up to 900 psia.

Generally, processes of the invention further comprise recovering from the resulting mixture on the permeate side a permeate product enriched in one or more hydrocarbon compounds over that of the depleted mixture on the first side. Processes of the invention include integration of perm-selective separations with purified product recovery operations, for example solid-bed selective sorption, fractional distillation, extractive distillation, solvent extraction and/or fractional crystallization.

Useful perm-selective hollow fiber membranes of the invention comprise a polymer derived from trimellitic anhydride chloride and one or more carbocyclic aromatic primary diamine, typically followed by final heat treatment after hollow fiber formation.

In other aspects, the invention provides a process for recovery of one or more hydrocarbon compounds in admixture with other organic compounds which process comprises: (a) contacting a fluid mixture comprising two or more hydrocarbon compounds each having a different boiling point temperature, with the non-permeate sides of a plurality of hollow fiber membranes comprising long-chain polymeric molecules in which recurring amide and imide linkages are part of the main polymer chain; and (b) selectively permeating at least one hydrocarbon compound of the mixture through the membrane to the permeate sides that are opposite the non-permeate sides, thereby exhibiting a separation factor of one aromatic hydrocarbon over another compound in a range upward from about 1.5.

The invention provides a process for recovery of one or more aromatic hydrocarbon compound from a fluid mixture that predominately comprises hydrocarbon compounds having at least 5 carbon atoms, that includes at least one aromatic hydrocarbon compound. Such fluid mixtures can comprise para-xylene and least one other C₈ aromatic compound. More particularly, invention provides a process for recovery of para-xylene from fluid mixtures that comprise the three isomers of xylene and optionally ethylbenzene, and the permeation advantageously exhibits a separation factor for pX/mX of at least 2.5.

In processes of the invention, selective permeations are beneficially carry out at temperatures in a range downward from about 220° C. to about 70° C. and feed pressures up to 900 psia, and thereby exhibit a para-xylene permeability in a range upward from 0.1 Barrer, and at least 0.5 Barrer for best results.

In another aspect, the invention provides a process for recovery of one or more aromatic hydrocarbon compound in admixture with other organic compounds which process comprises: providing a perm-selective, hollow fiber membrane, made by a method which comprises: preparing an extrudable spinning solution comprising a poly(amide)imide polymer, which in the form of a membrane exhibits a Stability Rating of a level 3 pass, and a solvent system containing at least one organic compound; extruding the spinning solution from an annular spinneret through an air gap into a quench bath containing water as a predominate component, while using a bore fluid, to from the hollow fiber; and drying the hollow fiber; contacting a fluid mixture comprising two or more aromatic hydrocarbon compounds, with a first side of a perm-selective, hollow fiber membrane comprising long-chain polymeric molecules in which recurring amide and imide linkages are part of the main polymer chain; and selectively permeating at least one aromatic hydrocarbon compound of the mixture through the membrane to a permeate side that is opposite the first side, thereby exhibiting a separation factor of one aromatic hydrocarbon over another compound in a range upward from about 1.5.

The extrudable spinning solution is a homogeneous solution made by dissolving polymer into on or more organic solvents which advantageously include dichloromethane, N-methyl-2-pyrrlidone, dimethylformamide, diethylformamide, dimethylacetamide, diethyl formamide, diethylacetamide, dimethyl sulfoxide, morpholine, dioxane, and the like. The bore fluid typically comprises water and one or more miscible organic solvent, such as N-methyl-2-pyrrolidone, and the like.

The perm-selective separations of the invention comprise of one or more devices using polymeric perm-selective membrane devices to separate a meta-xylene enriched stream from fluid mixtures of C₈ aromatics thereby producing a fluid comprising the remaining aromatic compounds which advantageously includes para-xylene. Processes of the invention are particularly useful for recovery of very pure meta-xylene and/or para-xylene products from liquid mixtures even containing ethylbenzene as well as the three xylene isomers.

Processes of the invention are particularly useful in processes for treatment of a mixture comprised of one or more products from reforming reactions, catalytic cracking reactions, hydro-processing reactions, para-selective toluene disproportion, a C₆ to C₁₀ aromatics trans-alkylation reaction, pyrolysis gasoline and/or methylation of benzene and/or toluene.

This invention is particularly useful towards separations involving organic compounds, in particular compounds which are difficult to separate by conventional means such as fractional distillation alone. Typically, these include organic compounds that are chemically related as for example substituted aromatic compounds of similar carbon number.

Other embodiments and objects of the present invention encompass details about feed mixtures, and operating conditions all of which are hereinafter disclosed in the following discussion of each of these facets of the present invention.

For a more complete understanding of the present invention, reference should now be made to the embodiments illustrated in greater detail in the accompanying drawing and described below by way of examples of the invention.

### BRIEF DESCRIPTION OF THE DRAWINGS

The invention is hereinafter described in detail with reference to the accompanying drawings which illustrate poly(amide)imide structure and synthesis.

FIG. 1 illustrates a general poly(amide)imide structure.

FIG. 2 illustrates an amide (“tail”) and imide (“head”) linkages.

FIG. 3 illustrates complete imidization synthesis.

### GENERAL DESCRIPTION

Any polymeric membrane which under a suitable differential of a driving force exhibits a permeability and other characteristics suitable for the desired separations may be used. For example, membrane devices for separations according to the invention may utilize a plurality of perm-selective membranes which under a suitable differential of a driving force exhibit a permeability of at least 0.1 Barrer for at least one of the isomers of xylene or ethylbenzene. Suitable membranes may take the form of a homogeneous membrane, a composite membrane or an asymmetric membrane.

The perm-selective polymeric membrane materials are used in separation processes wherein a fluid mixture contacts the upstream side of the membrane, resulting in a permeate mixture on the downstream side of the membrane with a greater mole fraction of one of the components than the composition of the original mixture. In some applications, a pressure differential is maintained between the upstream and downstream sides thereby providing a driving force for permeation.

Performance is characterized by the flux of a component across the membrane. This flux can be expressed as a quantity called the permeability (P), which is a concentration- and thickness-normalized flux of a given component. Separation of components is achieved by membrane materials that permit a faster permeation rate for one component (i.e., higher permeability) over that of another component. The efficiency of the membrane in enriching a component over another component in the permeate stream can be expressed as a quantity called selectivity. Selectivity can be defined as the ratio of the permeabilities of the gas components across the membrane (i.e., P_{A}/P_{B}, where A and B are the two components). A membrane's permeability and selectivity are properties of the membrane material itself, and thus these properties are ideally constant with feed concentration, flow rate and other process conditions. However, permeability and selectivity are both temperature-dependent. It is desired to employ membrane materials with a high selectivity (efficiency) for the desired component, while maintaining a high permeability (productivity) for the desired component.

If additional purification is desired, the product in the permeate stream can be passed through additional membranes, and/or the product can be purified via distillation and/or operations using techniques well known to those of skill in the art. Typically, membrane systems may consist of many modules connected in various configurations (See, for example, U.S. Pat. Nos. 6,830,691 and 6,986,802 the contents of which are hereby incorporated by reference for background and review). Modules connected in series offer many design possibilities to purify the feed, permeate, and residue streams to increase the separation purity of the streams and to optimize the membrane system performance.

Membranes useful for the separation of to C₈ aromatics in accordance with the invention include polymeric membrane systems. In such membrane systems, molecules permeate through the membrane. During permeation across the polymeric membrane, different molecules are separated due to the differences of their diffusivity and solubility within the membrane matrix. Not only does molecular shape influence the transport rate of each species through the matrix but also the chemical nature of both the permeating molecules and the polymer itself.

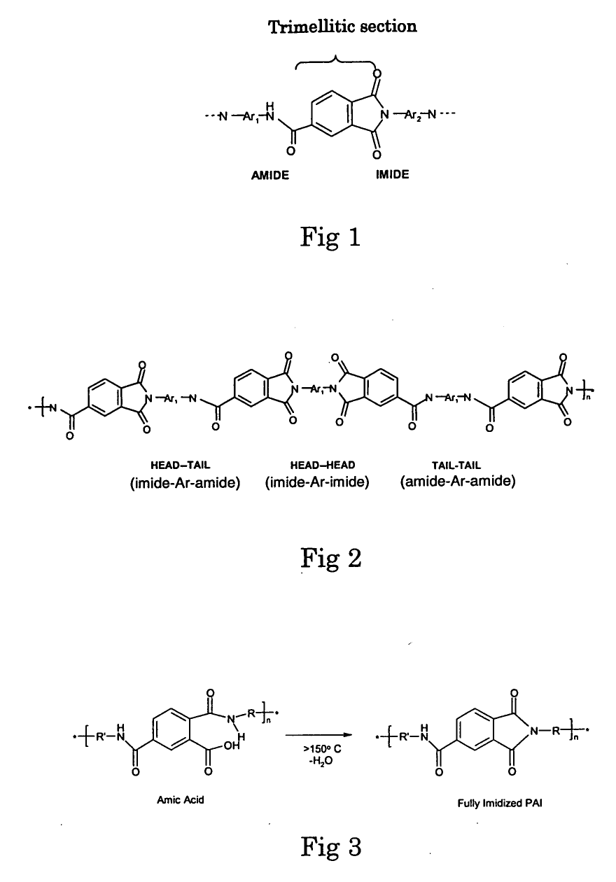

Advances in polymeric membranes make them attractive candidates for separation of aromatic compounds since they do not depend on easily poisoned metal complexes to achieve the separation. For example, film forming poly(amide)imide materials are derived from trimellitic anhydride and aromatic diamines. FIG. 1 illustrates a general poly(amide)imide structure where Ar₁ and Ar₂ represent aromatic groups that can be connected to the trimellitic section via an amide (“tail”) or imide (“head”) linkage as illustrated in FIG. 2.

The distribution of the linkages has a role in determining properties of poly(amide)imide membranes. Commercial products typically have a random distribution of head-tail, head-head and tail-tail linkages due to the method of preparation, whereas more region-specific synthetic materials have also been developed. While poly(amide)imide materials have not been investigated as much as polyimides in membrane applications, primarily because of the much lower permeability due to the amide group, the amide group is also responsible for better mechanical properties and improved chemical resistance compared to polyimides. Poly(amide)imide materials typically are soluble in aprotic polar solvents such as NMP, DMAC, and DMF, and occasionally in THF. They are glassy, amorphous materials benefically with high glass transition temperatures (Tg) of about 250° C. allowing use at elevated temperatures approaching the glass transition temperature of the poly(amide)imide membranes, for example an upper use temperature of about 200° C.

As with polyimides, there is often some “amic acid” present in the poly(amide)imide polymer. Nearly complete imidization (>95%) can be realized by either heating, as illustrated in FIG. 3, or using chemical reagents (e.g., triethylamine/acetic anhydride) in order to attain the ultimate final properties. Average molecular weight of poly(amide)imide polymers are usually determined by the inherent viscosity using an Ubbelohde viscometer.

Preparation of useful film forming poly(amide)imide materials is disclosed in U.S. Pat. No. 3,920,612 which is incorporated herein by reference in its entirety. Essentially equimolar amounts of a carbocyclic aromatic primary diamine and an acyl halide derivative of trimellitic anhydride are reacted under essentially anhydride conditions and for a period of time and at a temperature controlled to produce a polymer with free carboxyl groups and amide groups available for further reaction. Cured poly(amide)imide polymers are formed by heating of the soluble polymers at a temperature above 150° C. sufficient to effectively and substantially convert such carboxyl and amide groups to imide groups. Typically the condensation reactions are carried out in a solvent, such as DMAC, at temperatures in a ranger of from about 40 to 50° C., and then the polymer is precipitated in acetone or water. Higher molecular weight product that can more easily be drawn in to hollow fibers is made by lowering the initial temperature and adding a base such as CaO to neutralize the HCl. The poly(amide)imide polymers made in this way have roughly a random distribution of linkages since the diamine reacts nearly equally with both the anhydride and the acid chloride groups.

A particularly useful poly(amide)imide membrane material is made by adding TMACl to a mixture of two diamines—4,4′-oxydianiline (ODA) and m-phenylenediamine (m-PDA). The poly(amide)imide materials made with p-methylenedianiline (MDA) as the only aromatic diamine and are not as thermally stable as the two diamine series since the methylene group is more prone to free-radical degradation/cross-linking reactions in the presence of oxygen at high temperatures.

Another class of useful poly(amide)imide membrane materials is discussed in U.S. Pat. No. 4,505,980 which is incorporated herein by reference in its entirety. This class of polyamide-imide materials is prepared by reacting an aromatic diisocyanate and a tricarboxylic acid anhydride, typically trimellitic acid anhydride, in the presence of a basic solvent. The aromatic diisocyanate used includes, for example, tolylene diisocyanate, xylylene diisocyanate, 4,4′-diphenylether diisocyanate, naphthalene-1,5-diisocyanate, 4,4′-diphenylmethane diisocyanate, isophorone diisocyanate, 1,6-hexamethylene diisocyanate, cyclohexane diisocyanate, etc. When heat resistance and the like are taken into consideration, it is preferable to use 4,4′-diphenylmethane diisocyanate or tolylene diisocyanate. If necessary, there may be co-used aliphatic diisocyanates such as 1,6-hexamethylene diisocyanate, isophorone diisocyanate and the like, alicyclic diisocyanates, trimers thereof, isocyanurate-ring-containing polyisocyanates obtained by trimerization reaction of the aforesaid aromatic diisocyanates, polyphenylmethyl polyisocyanates, e.g., a phosgenated condensate of aniline and formaldehyde, etc. In particular, isocyanurate-ring-containing polyisocyanates obtained by trimerization reaction of tolylene diisocyanate or 4,4′-diphenylmethane diisocyanate are particularly useful.

If desired, polycarboxylic acids or acid anhydrides thereof other than the tricarboxylic acid anhydride described above may also be co-used. These include, for example, trimellitic acid, trimesic acid, tris(2-carboxyethyl)isocyanurate, terephthalic acid, isophthalic acid, succinic acid, adipic acid, sebacic acid, dodecanedicarboxylic acid and the like.

Dianhydrides of tetrabasic acids may be used, for example, aliphatic and alicyclic tetrabasic acids such as 1,2,3,4-butanetetracarboxylic acid, cyclopentanetetracarboxylic acid, ethylenetetracarboxylic acids, bicyclo-[2,2,2]-octo-(7)-ene-2:3,5:6-tetracarboxylic acid; aromatic tetrabasic acids such as pyromellitic acid, 3,3′,4,4′-benzophenonetetracarboxylic acid, bis(3,4-dicarboxyphenyl)ether, 2,3,6,7-naphthalenetetracarboxylic acid, 1,2,5,6-naphthalenetetracarboxylic acid, ethylene glycol bistrimellitate, 2,2′-bis(3,4-biscarboxyphenyl)propane, 2,2′,3,3′-diphenyltetracarboxylic acid, perylene-3,4,9,10-tetracarboxylic acid, 3,4-dicarboxyphenylsulfonic acid; and heterocyclic tetrabasic acids such as thiophene-2,3,4,5-tetracarboxylic acid, and pyrazinetetracarboxylic acid.

When the aromatic diisocyanate and the tricarboxylic acid anhydride are reacted in approximately equimolar amounts, a polyamide-imide resin having a sufficiently high molecular weight is obtained at the time of curing, and shows the best heat resistance and flexibility. Although the diisocyanate compound may be added in slightly excessive amount of moles in consideration of the fact that a small amount of water contained as an impurity in the reaction solvent reacts with isocyanate groups. For best results, the amount of the aromatic diisocyanate compound must be not more than 1.1 moles per mole of the tricarboxylic acid anhydride.

Useful basic solvents are those which are substantially inert to the aromatic diisocyanates. For example, N-methylpyrrolidone, N-methyl-caprolactam, N,N-dimethylformamide, N,N-dimethylacetamide, hexamethylphosphoneamide, and dimethylsulfoxide can be used. As a synthesis solvent for the aromatic diisocyanate and the tricarboxylic acid anhydride, N-methylpyrrolidone is preferred. As a dilution solvent used after the reaction, dimethylformamide is preferred.

A particularly useful class of poly(amide)imide membrane materials is described in U.S. Pat. No. 5,124,428 which is incorporated herein by reference in its entirety. Generally the poly(amide)imide materials described included polymers obtained from reactants which comprise the reactant pair trimellitic anhydride chloride (TMAC) and toluene diamine (TDA); or the reactant pair trimellitic anhydride (TMA) and toluene diisocyanate (TDI). These poly(amide)imide materials are further characterized in terms of specified inherent viscosity and molecular weight values which are critical for obtaining resin which can be solution-spun into heat-resistant hollow-fibers of high quality.

U.S. Pat. No. 5,124,428 further provided a process for manufacturing poly(amide)imide materials from trimellitic anhydride chloride and toluene diamine by:

(a) reacting, in a polar organic solvent in the presence of a suitable acid scavenger, trimellitic anhydride chloride and toluene diamine in a mole ratio of about 0.95:1 to 1.01:1 to obtain a solution comprising a dissolved polymeric condensation product containing amide, imide and amic-acid linkages, wherein the reaction is carried out essentially to completion under conditions of time and temperature such that polymer linkages derived from anhydride moieties are predominantly amic-acid linkages;

(b) heating the solution obtained in (a) under conditions of time and temperature sufficient to obtain a solution wherein the condensation product present in the solution has undergone conversion to a polyamide-imide such that greater than about 90 percent of the polymer linkages derived from anhydride moieties are imide linkages; and

(c) continuing heating of the poly(amide)imide solution obtained in step (b) until the poly(amide)imide has an inherent viscosity of from about 0.3 to about 1.3 dL/g.

Suitable poly(amide)imide materials are also made from trimellitic anhydride and toluene diisocyanate by reacting, in a solvent in the presence of a suitable catalyst, toluene diisocyanate and trimellitic anhydride in a mole ratio of from about 0.95:1 to about 1.01:1 at a temperature in the range of from about 150° C. to about 200° C. to obtain a solution comprising the dissolved amide-imide polymeric condensation product of these reactants in the polar solvent. For best results, the reaction is conducted until at least about 90 percent of polymer linkages derived from anhydride groups are imide linkages.

A Patent that discusses useful poly(amide)imide material having head to tail backbone is U.S. Pat. No. 6,433,184 which is also incorporated herein by reference in its entirety. More particularly, the patent discloses a class of poly(amide)imide materials having head-to-tail regularity to provide excellent heat and chemical resistance, physical and mechanical properties, processability, and gas permeability and selectivity.

These poly(amide)imide materials are obtained by direct polymerization, in the presence of dehydrating catalyst, of an amine compound having a nitro group with a carboxylic acid anhydride, such as trimellitic anhydride thereby forming a precursor imide. The nitro group is then hydrogenated to form an amine that is the condensed with the tail end carboxyl group.

A useful class of membranes for separation embodiments is a type of composite membranes which comprise a microporous support, onto which the perm-selective layer is deposited as an ultra-thin coating. Another useful class is asymmetric membrane in which a thin, dense skin of the asymmetric membrane is the perm-selective layer. Both composite and asymmetric membranes are known in the art. The form in which the membranes are used in the invention is not critical. They may be used, for example, as flat sheets or discs, coated hollow fibers, spiral-wound modules, or any other convenient form.

Advantageously the hollow fiber polymer membrane is a composite material comprising an effective skin layer and a porous support. The porous support material can be the same or different polymer as the membrane. Generally the porous support is an inexpensive porous polymer. In a composite hollow fiber polymer membrane the porous support layer can be either the inside layer or the outside layer. Typically the porous support layer is the inside layer in this embodiment and the “skin” layer is on the outside of the hollow fiber.

Hollow fiber membranes are discussed in U.S. Pat. No. 6,562,110 and U.S. Pat. No. 6,585,802 which are incorporated herein by reference in their entirety. The high permeability and selectivity of hollow fiber membranes, beneficial for practice of the present invention, depends at least in part upon control of the molecular weight of the polymer material. Control of molecular weight is needed to form hollow fiber membranes that are not too brittle and exhibit an effective skin layer. Generally for processes of the present invention, the average polymer molecular weight is between about 20,000 and about 200,000, typically between about 40,000 and about 160,000, and depending upon the separation desired, between about 60,000 and about 120,000 for best results.

Suitable types of membrane devices include spiral-wound, plate-and-frame, and tubular types. The choice of the most suitable membrane module type for a particular membrane separation must balance a number of factors. The principal module design parameters that enter into the decision are limitation to specific types of membrane material, suitability for high-pressure operation, permeate-side pressure drop, concentration polarization fouling control, permeability of an optional sweep stream, and last but not least costs of manufacture.

Hollow-fiber membrane modules are used in two basic geometries. One type is the shell-side feed design, which has been used in hydrogen separation systems and in reverse osmosis systems. In such a module, a loop or a closed bundle of fibers is contained in a pressure vessel. The system is pressurized from the shell side; permeate passes through the fiber wall and exits through the open fiber ends. This design is easy to make and allows very large membrane areas to be contained in an economical system. Because the fiber wall must support considerable hydrostatic pressure, the fibers usually have small diameters and thick walls, e.g. 100 μm to 200 μm outer diameter, and typically an inner diameter of about one-half the outer diameter.

A second type of hollow-fiber module is the bore-side feed type. The fibers in this type of unit are open at both ends, and the feed fluid is circulated through the bore of the fibers. To minimize pressure drop inside the fibers, the diameters are usually larger than those of the fine fibers used in the shell-side feed system and are generally made by solution spinning. These so-called capillary fibers are used in ultra-filtration, pervaporation, and some low- to medium-pressure gas applications.

Concentration polarization is well controlled in bore-side feed modules. The feed solution passes directly across the active surface of the membrane, and no stagnant dead spaces are produced. This is far from the case in shell-side feed modules in which flow channeling and stagnant areas between fibers, which cause significant concentration polarization problems, are difficult to avoid. Any suspended particulate matter in the feed solution is easily trapped in these stagnant areas, leading to irreversible fouling of the membrane. Baffles to direct the feed flow have been tried, but are not widely used. A more common method of minimizing concentration polarization is to direct the feed flow normal to the direction of the hollow fibers. This produces a cross-flow module with relatively good flow distribution across the fiber surface. Several membrane modules may be connected in series, so high feed solution velocities can be used. A number of variants on this basic design have been described, for example U.S. Pat. Nos. 3,536,611 in the name of Fillip et al., 5,169,530 in the name of Sticker et al., 5,352,361 in the name of Parsed et al., and 5,470,469 in the name of Beckman which are incorporated herein by reference each in its entirety. The greatest single advantage of hollow-fiber modules is the ability to pack a very large membrane area into a single module. U.S. Pat. No. 5,266,197 in the name of Jitsumi Tahata and Isamu Yamamoto describes hollow fiber membranes, suitable for blood purification. Their hollow fiber membranes were made of a polyamidimide having a particular structure which provided useful micropores.

Integrated processes that comprises separations by means of one or more devices using perm-selective polymeric membranes coupled with recovery of purified products by means of fractional crystallization and/or selective sorption are described in copending U.S. patent application Ser. Nos. 10/769,538 and 10/769,539 which are incorporated herein by reference in their entirety. These processes are particularly useful for recovery of a very pure aromatic isomer when processing aromatic starting materials, for example, a pure para-xylene product from liquid mixtures even containing ethylbenzene as well as the three xylene isomers.

Sources of xylenes in substantial quantities include certain virgin and reformed petroleum naphthas, pyrolysis gasoline, coke oven light oils, and hydro-cracking heavy aromatics such as gas-oil and LCCO (light cat cycle oil). When removed from typical petroleum-derived feedstocks, para-xylene is found in mixtures with other C₈ aromatics; namely: meta-xylene, ortho-xylene, and ethylbenzene. Generally, processes of the invention recover a very pure isomer of xylene from distillate fractions containing the xylene isomers, ethylbenzene, and paraffins.

While many sources of C₈ aromatics may be fed to the process, in a typical C₈ fraction from a naphtha reformer and aromatics recovery unit the mixture contains approximately 15 percent ethylbenzene, 22 percent para-xylene, 50 percent meta-xylene and 22 percent ortho-xylene and varying amounts of saturated and unsaturated, linear and cyclic hydrocarbons. Other sources of suitable C₈ aromatics include transalkylation of toluene and C₈/C₁₀ reformate, and toluene disproportionation.

In view of the features and advantages of the processes for recovery of value added products from a fluid admixture of hydrocarbon compounds, least one of which is an aromatic hydrocarbon compound, in accordance with this invention as compared to other membrane reactors previously proposed and/or employed for the separation, the following examples are given.

### EXAMPLES

### General

Polymer membrane films were tested by pervaporation in a high temperature pervaporation or vapor-phase apparatus designed to operate at temperatures in excess up to 200° C. A microprocessor (VWR) controlled vacuum oven was modified to hold the pervaporation apparatus. The system was connected to pressure transducer and data logger for continuous measurement of system downstream pressure. As well, the system had the ability to interface with a gas chromatograph (HP 6890) for downstream compositional analysis.

Polymer membrane films were placed in a specially designed cell that held about 500 mL of agitated feed solution on the upstream of the membrane. The downstream was subject to vacuum, which induces a chemical potential driving force across the membrane, thus causing a permeation flux. At steady state, composition of the permeate was analyzed and compared to the feed to obtain a composition selectivity via gas chromatography.

A. Dope Preparation: Depending on the desired film casting method, one of two types of polymer casting “dopes” is required. Films can be cast either by dropping the dope onto the substrate through a syringe or by pouring the dope onto a substrate and drawing it into a thin film with a casting knife. Only dilute dopes (less than about 5 percent solids) can be dropped through a syringe, while more concentrated, highly viscous dopes must be drawn with a knife.

Regardless of whether a dilute or concentrated casting dope is desired, the first step is to dry the materials in a vacuum oven overnight. Polymers are normally dried between 100° C. and 140° C. To prepare a dilute polymer solution, the desired amount of polymer is placed in a clean glass bottle, an appropriate filtered solvent is added to give a 1 to 2 percent polymer solution, and the solution is allowed to dissolve. Dissolution usually occurs within a few hours, and sometimes assisted with heat. When casting films, an approximately 20 percent polymer solution is required to give the desired film thickness of 1 to 2 mils using the casting knife of 8 to 16 mil clearance. Concentrated polymer solutions are placed on a mechanical roller to dissolve without entraining excess air. This polymer solution is generally rolled overnight to assure complete dissolution.

B. Film Preparation: Once the casting dope is ready, films are cast using one of two methods. It is necessary to cast solutions with low volatility solvents on glass in the vacuum oven or on a hotplate to remove the solvent. All films are covered with an inverted pan or funnel to keep dust from settling on the nascent films.

To cast a pure polymer film from a dilute solution, the solution is first poured into a glass syringe with a 0.2 μm PTFE filter attached to the tip. The casting dope is dropped onto the substrate into a circular stainless steel or PTFE mold. The amount of dope required is determined by the desired film thickness, the area of the stainless steel casting mold, the dope concentration, and the density of the resulting membrane. Should any air bubbles develop in the nascent film, they can be pushed up against the edge of the stainless steel casting ring with the tip of the syringe.

Viscous dopes are cast by first pouring them onto the desired casting substrate. A casting knife (Paul N. Gardner & Co.; Pompano Beach, Fla.) with an 8, 10, 12, or 16 mil clearance is then used to draw the dope into a film of uniform thickness.

Poly(amide)imide separation membranes used in these examples of the invention are believed to be condensation products of trimellitic anhydride chloride (TMAC) condensed with two diamines, 4,4′-oxydianiline (ODA) and m-phenylenediamine (m-PDA), available as TORLON®4000T from Solvay Advanced Polymers, Alpharetta, Ga., USA.

All poly(amide)imide films, regardless of preparation method, were dried, typically from (N-methylpyrolidone (NMP), after formation. Drying was normally completed at elevated temperatures (about 250° C.). Some films were annealed above the glass transition temperature, Tg, of the poly(amide)imide. This step is to remove residual solvent to extremely low levels, and reduces any effect residual solvent may play in transport analysis.

C. Fiber Preparation: In order to create fibers via non-solvent induced phase separation, a viscous homogeneous polymer solution, known as a dope, is extruded through an annular die (spinneret) at low temperatures (30-60° C.). The nascent fiber is then subjected to an environment that induces phase separation, either through the preferential evaporation of solvents over non-solvents from the dope or immersion in a non-solvent quench bath. These spinning techniques can be sub-divided further, characterized by the environments encountered by the nascent fiber after exiting the spinneret.

In somewhat greater detail, once a dope has been prepared, it is co-extruded with a bore fluid through an annular die known as a spinneret. The bore fluid can be gas or liquid, and is used to prevent the nascent hollow fiber from collapsing. In the dry-jet portion of the spinning, the extrudate, or nascent fiber, passes through an air gap. The air gap can be controlled for length, humidity, temperature, and even composition (nitrogen, air, vapors, etc.).

While the nascent fiber passes through the air gap, it will have various interactions with the air gap. For example, volatile components from the dope may evaporate, increasing the concentration of polymer in the outside layers. At the same time, non-solvent vapors, primarily water vapor, can be absorbed by the solution.

After passing through the air gap, the nascent fiber enters the liquid quench bath (wet-quench). This is typically an aqueous bath, but any liquid that acts as a non-solvent for the polymer system can be used as a coagulant. When the nascent fiber enters the quench bath, non-solvent diffuses into the fiber, while solvent will often diffuse out into the bath. Phase separation is often very rapid at this point, as the spin dope rapidly de-mixes into polymer rich and polymer lean phases. The polymer rich phases will eventually form the solid fiber structure, while the polymer lean phase will be washed out, leaving behind the pores in the fibers substructure.

Once solidified, the fiber will typically pass through several guides on its way to a take-up device, often as simple as a rotating drum around which the fibers are wound. By controlling the take-up speed and extrusion rate, the draw ratio (ratio of take-up and extrusion rates) and diameter of the fibers can be controlled.

Polymer membranes were tested as flat films sealed by O-rings between two metal plates. The membranes were tested in a continuous flow, automated pilot unit for up to 20 days. On the feed side of the membrane, hydrocarbon mixtures were contacted as liquids or vapors at the desired temperature pressure and flow rate. Hydrocarbons passing through the membrane were swept by nitrogen flow at atmospheric pressure and analyzed by on-line gas chromatography. From the flow rate of the sweep gas and the concentration of hydrocarbons the normalized flux per area (kg-μm/m²-hr) was calculated. The separation factor was calculated by taking the ratio of the concentration in the permeate divided by the concentration of each component in the feed. The hydrocarbon feeds, feed pressures, membrane temperatures, separation factors and permeation rates are given in the tables of results.

### Stability Rating

Prior to evaluation according to the test program described above, a sample of each membrane material exhibited a Stability Rating of at least a level 1 pass. Stability Ratings were determined after exposure, at elevated temperatures, of membrane materials to a flow of mixed xylenes in helium diluent. Conditions of exposure are shown in Table I. After exposure, membranes were rated passed, or failed, based upon physical condition. Examples of materials that were rated as failed at level 1 include a commercially available polyetherimide, (ULTEM® from GE, ______, USA) and a polyethylene film. A silicon rubber membrane material was rated passed at level 1, but at level 2 failed.

TABLE I

| | Level 1 | Level 2 | Level 3 |
|---|---|---|---|
| Item | Conditions | Conditions | Conditions |
| Temperature | 100° C. | 150° C. | 200° C. |
| Feed, mL/hr | 0.31 | 5.0 | 10.0 |
| Helium Flow, | 100 | 15 | 15 |
| cc/min | | | |
| Pressure, | 15 | 15 | 0 |
| psig | | | |
| Time, hr | 6 or 18 | 6 or 18 | 6 or 18 |

### Comparative Example A

In this example the test program described above was used to evaluate a poly(amide)imide membrane formed from the condensation product of DAM and 6FPDA for separation of an equal molar admixture of the three isomers of xylene, at a feed rate of 5 mL/hr. Runs for this example were carried out at permeate side pressures of from 15 psia and an oven temperature of 75° C. This poly(amide)imide membrane material exhibited a Stability Rating of a level 2 pass, however at 100° C. the membrane failed, and the best separation factor observed was only 1.34 for para to ortho xylenes. Results are summarized in Table II.

TABLE II

| | | Separation | Separation | |
|---|---|---|---|---|
| | TEMP, | factor: | factor: | Permeation Rate |
| RUN | ° C. | pX/oX | pX/mX | (kg-μm/m²-hr)) |
| 12 | 75 | 1.34 | 1.13 | 0.71 |
| 13 | 100 | — | — | Membrane Failed |

pX is para-xylene, oX is ortho-xylene, and mX is meta-xylene

### Example 1

In this example of the invention, a poly(amide)imide membrane material, which had exhibited a Stability Rating of a level 3 pass and a glass transition temperature (Tg) of about 250° C., was evaluated for separation of a equal admixture of the three isomers of xylene (dimethylbenzenes) at a feed rate of 5 mL/h. This poly(amide)imide membrane was made of TORLON® 4000T and sourced from Solvay Advanced Polymers, Alpharetta, Ga., USA).

Runs for this example were carried out at oven temperatures of 100° C. and 150° C. and permeate side pressures of from 15 psia to 150 psig. Observed results are summarized in Table III.

### Example 2

In this example of the invention, a poly(amide)imide membrane material, which had exhibited a Stability Rating of a level 3 pass and glass transition temperatures (Tg) of about 250° C., was evaluated for separation of a 30:30:30:10 a admixture of the three isomers of xylene, and ethylbenzene, at a feed rate of 3 mL/hr. This poly(amide)imide membrane was made of TORLON® 4000T and sourced form Solvay Advanced Polymers, Alpharetta, Ga., USA). The membrane, had a nominal thickness of 1.5 mls. Runs for this example were carried out at oven temperatures of 100° C. to 170° C. and permeate side pressures of from 15 psia. Observed results are summarized in Table IV.

TABLE III

| | | | Separation | Separation | |
|---|---|---|---|---|---|
| | TEMP, | Pressure | factor: | factor: | Permeation Rate |
| RUN | ° C. | (psig) | pX/mX | pX/oX | (kg-μm/m²-hr) |
| 3 | 100 | 15 | 1.64 | 2.21 | 0.47 |
| 4 | 110 | 15 | 1.55 | 2.07 | 0.741 |
| 5 | 110 | 29 | 1.57 | 1.98 | 0.795 |
| 6 | 110 | 50 | 1.73 | 2.20 | 0.779 |
| 7 | 120 | 50 | 1.61 | 2.16 | 1.174 |
| 8 | 130 | 50 | 1.55 | 2.07 | 1.676 |
| 9 | 130 | 100 | 1.50 | 1.96 | 1.506 |
| 10 | 150 | 100 | 1.63 | 2.35 | 0.588 |
| 11 | 130 | 100 | 1.72 | 2.77 | 0.206 |

pX is para-xylene,
oX is ortho-xylene, and
mX is meta-xylene

TABLE IV

| | Separation | Separation | Separation | |
|---|---|---|---|---|
| TEMP, | factor: | factor: | factor: | Permeation Rate |
| ° C. | pX/mX | pX/oX | pX/EB | (kg · μm/m² · hr) |
| 130 | 1.52 | 2.16 | 1.42 | 0.64 |
| 100 | 1.64 | 2.33 | 1.53 | 0.87 |
| 150 | 1.50 | 2.20 | 1.62 | 0.69 |
| 130 | 1.58 | 2.41 | 1.64 | 0.20 |
| 170 | 1.54 | 2.02 | 1.53 | 0.43 |
| 150 | 1.47 | 2.22 | 1.51 | 0.17 |
| 130 | 1.65 | 2.06 | 1.45 | 0.09 |

pX is para-xylene,
oX is ortho-xylene, and
mX is meta-xylene

### Example 3

In this example of the invention, a poly(amide)imide membrane material, which had exhibited a Stability Rating of a level 3 pass, was evaluated for separation of a 30:30:30:10 a admixture of the three isomers of xylene, and ethylbenzene, at a feed rate of 3 mL/hr. This poly(amide)imide membrane was made of TORLON® 4000T, and had a nominal thickness of 1.5 mils. Runs for this example were carried out at permeate side pressures of from 15 psia and oven temperatures of 100° C. for 6 days and 130° C. for 3 days. Observed results are summarized in Table V.

TABLE V

| | Separation | Separation | Separation | |
|---|---|---|---|---|
| TEMP, | factor: | factor: | factor: | Permeation Rate |
| ° C. | pX/mX | pX/oX | pX/EB | (kg-μm/m²-hr) |
| 100 | 1.45 | 1.97 | 1.39 | 0.92 |
| 130 | 1.44 | 1.94 | 1.39 | 1.68 |

### Example 4

This example of the invention evaluated a poly(amide)imide membrane, formed from the condensation product of 2,6-diaminomesitylene (DAM) and p-methylenedianiline (MDA), for separation of an equal molar admixture of the three isomers of xylene, at a feed rate of 5 mL/hr. The poly(amide)imide membrane material exhibited a Stability Rating of a level 1 pass. Runs for this example were carried out at permeate side pressures of from 15 psia and an oven temperature in a range from 60° C. to 130° C. Observed results are summarized in Table VI.

### Example 5

This example of the invention evaluated three poly(amide)imide membranes for separation of a 30:30:30:10 a admixture of the three isomers of xylene, and ethylbenzene, in a constantly agitated 450 mL volume on the upstream side of the membrane. These poly(amide)imide membranes were made of TORLON® 4000T, which exhibited a Stability Rating of a level 3 pass, had a nominal thickness of 1.5 mils. The membranes, identified as 7a, 8a, and 10a, were annealed to 300° C. (Ramp to 300° C. at 10° C./min and cooled over 4 hours). Runs for this example were carried out at permeate side pressures of from 30 psia and oven temperatures of 210° C. Observed results are summarized in Table VII.

TABLE VI

| | | Separation | Separation | |
|---|---|---|---|---|
| | TEMP, | factor: | factor: | Permeation Rate |
| RUN | ° C. | pX/oX | pX/mX | (kg-μm/m²-hr) |
| 14 | 75 | 1.56 | 1.19 | 0.17 |
| 15 | 100 | 1.45 | 1.17 | 0.59 |
| 16 | 130 | 1.39 | 1.17 | 1.64 |
| 17 | 60 | 1.07 | 1.02 | 0.13 |

TABLE VII

| | Separation | Separation | Para-Xylene |
|---|---|---|---|
| | factor: | factor: | Permeabilit, |
| Film ID | pX/others | pX/oX | Barrer |
| 7a | 2.80 | 2.45 | 0.68 |
| 8a | 3.96 | 7.44 | 0.73 |
| 10a | 1.81 | 2.23 | 0.54 |

### Example 6

This example describes spinning a hollow-fiber and preparation of membrane separation modules. The poly(amide)imide used a condensation product of trimellitic anhydride chloride (TMAC) with two diamines, 4,4′-oxydianiline (ODA) and m-phenylenediamine (m-PDA). The poly(amide)imide was sourced from Solvay Advanced Polymers, Alpharetta, Ga., USA).

A spinning solution (dope) was composed following the compositions indicated on the weblink:

www.chbe.gatech.edu/faculty_staff/faculty/koros/group webPage/People/Madhava_kosuri.htm.

Specifically, the dope contained a homogenios solution of 27 wt % Torlon 4000 poly(amide)imide, 50 wt % N-methyl-2-pyrrolidone (NMP), 13 wt % tetrahydrofuran (THF) and 10 wt % ethanol. The dope was rolled in a sealed container for 5 days to ensure complete mixing. The dope was then allowed to degas for 24 hours before being poured into an ISCO® syringe pump, where it was again degassed for 24 hours.

As described in the dissertation of Zhou (Ref), the dope was extruded from an annular spinneret at 3 mL/min through an air gap into a quench bath filled with deionized water and taken up on a rotating drum at between 20 and 50 m/min. A solution consisting of 80 percent NMP with 20 percent water was used as the bore fluid. The hollow-fibers were kept wetted with deionized water while on the take-up drum. The hollow-fibers were cut from the drum with a razor to lengths of one meter and washed in deionized water for 72 hours.

After washing in water, the hollow-fibers were washed in baths of ethanol (3×30 min) and hexane (3×30 min). The hexane-wet fibers were allowed to air dry for one hour, and then dried under vacuum at 120° C. for one hour.

The hollow-fibers were heat treated by exposure to 150° C., for 25 hours under vacuum. They were subsequently potted into modules for evaluation of their selective permeation properties. The membrane modules consisted of one quarter inch stainless steel tubes with side entry and exit “tee” fittings and end fittings into which the hollow fibers have been sealed with an epoxy resin. The length of the modules is such that the hollow-fibers had a nominal length of 11.25 inches, although the length available for permeation was less due to the epoxy seal. The side “tee” fittings provide access to the external fiber surfaces while the end fittings expose the bore of each fiber for internal flow. The side fittings are centered along the length of the tube and are 5.25 inches apart.

### Example 7

This example of the invention evaluated permeation properties the hollow-fiber modules of Example 6 in an experimental unit, which was designed to evaluate membranes of various types for selectivity and flux. In general, feed mixtures were supplied, along with a diluent gas, to one side of the membrane in either liquid or vapor form, and a sweep gas was used to carry permeate into an on-line gas chromatograph.

The feed mixture used in testing of the poly(amide)imide hollow-fiber module was a mixture of methylcyclopentane, cyclohexane, n-heptane and benzene in equal amounts by weight. A flow of nitrogen diluent was mixed with the liquid feed to ensure vaporization at all conditions and to stabilize pressure control. The feed vapor was introduced to the membrane through a side fitting on the module, thus being exposed to the external surface of the fibers. Nitrogen was used as the “sweep gas” in this test and was directed through the bore of the fibers via the module's end fittings.

Test conditions: The nitrogen diluent flow rate was held constant at 100 sccm while the nitrogen seep flow was maintained at 20 sccm. The pressure on the permeate side of the membrane was nominally 1 atm (absolute) for the duration of the test. The liquid feed rate was set initially to 10 mL/hr and was reduced to 5 mL/hr after about three to seven days. This hollow-fiber module was tested for a total of approximately 19 days during which conditions were changed 16 times. In Table VIII, test conditions are presented with selected results showing only stable, lined-out results, due to the large amount of data acquired during the testing. Each row presents data collected at conditions different than the previous row and rows are arranged in chronological order. The feed rate initially was 10 mL/hr, but was reduced on day 7 to 5 mL/hr.

TABLE VIII

| | | Feed | | Separation | Separation | Separation |
|---|---|---|---|---|---|---|
| | T, | Press | Total | factor: | factor: | factor: |
| Day | ° C. | atm | Perm. | Bz/: MCP | Bz/CHEX | Bz/nC7 |
| 1 | 100 | 2.0 | 4.46 | 1.42 | 1.11 | 0.86 |
| 2 | 150 | 2.0 | 2.75 | 11.39 | 8.08 | 3.65 |
| 2.5 | 100 | 2.0 | 1.17 | 2.23 | 1.67 | 1.25 |
| 3 | 150 | 2.0 | 2.11 | 10.86 | 7.24 | 3.77 |
| 7 | 150 | 2.0 | 1.77 | 11.24 | 10.58 | 10.09 |
| 8 | 125 | 2.0 | 0.93 | 5.28 | 4.79 | 4.86 |
| 9 | 100 | 2.0 | 0.66 | 2.66 | 2.42 | 2.42 |
| 9.5 | 150 | 2.0 | 1.51 | 10.49 | 9.84 | 9.23 |
| 10 | 175 | 2.0 | 3.13 | 21.70 | 20.75 | 17.89 |
| 10.5 | 150 | 2.0 | 1.72 | 11.80 | 10.82 | 10.35 |
| 11 | 150 | 4.5 | 6.92 | 3.11 | 2.81 | 2.76 |
| 12 | 175 | 4.5 | 9.67 | 5.13 | 4.64 | 4.38 |
| 14 | 125 | 4.5 | 5.94 | 1.89 | 1.67 | 1.59 |
| 17 | 100 | 4.5 | 4.00 | 1.50 | 1.34 | 1.32 |
| 18 | 150 | 2.0 | 1.54 | 10.76 | 9.88 | 9.31 |
| 19 | 200 | 2.0 | 4.75 | 33.74 | 34.37 | 26.02 |
| 19.5 | 200 | 1.5 | 3.09 | 42.58 | 44.96 | 32.68 |

Total Perm is Permeation Rate in units of E-5 (kg-μm/m²-hr).
BZ is benzene,
MCP is methylcyclopentane,
CHEX is cyclohexane, and
nC7 is n-heptane.

### Comparative Example B

In this example the test program described above was used to evaluate a polyimide membrane for separation of a 50:50 admixture of benzene and cyclohexane at a feed rate of 5 mL/hr. The polyimide membrane tested was formed from a commercially available polyimide, (MATRIMID® from Vantico, Inc., Brewster, N.Y., USA) that is believed to be a condensation product of BTDA and DAPI. This polyimide membrane material exhibited a Stability Rating of a level 3 pass. Runs were carried out at oven temperatures from 75° C. to 100° and feed pressures of 15 and 29 psig. Observed results are summarized in Table IX.

TABLE IX

| | | Pressure | Separation factor | Permeation Rate |
|---|---|---|---|---|
| RUN | TEMP | (psig) | BZ/CHEX | (kg-μm/m²-hr) |
| 1 | 75° C. | 15 | 11.4 | 0.55 |
| 2 | 85° C. | 15 | 7.3 | 1.21 |
| 3 | 85° C. | 29 | 6.9 | 1.30 |
| 4 | 100° C. | 29 | 4.6 | 2.87 |

BZ is benzene and CHEX is cyclohexane.

### Example 8

This example of the invention evaluated a poly(amide)imide membrane for separation of a 50:50 admixture of benzene and cyclohexane at a feed rate of 5 mL/hr. This poly(amide)imide membranes was made of TORLON® 4000T, which exhibited a Stability Rating of a level 3 pass, had a nominal thickness of 1.5 mils. Runs for this example were carried out at oven temperatures of 100° C. and 115° C. and feed pressures of 15 psig. Observed results are summarized in Table X.

TABLE X

| | | Pressure | Separation factor | Permeation Rate |
|---|---|---|---|---|
| RUN | TEMP | (psig) | BZ/CHEX | (kg-μm/m²-hr) |
| 1 | 100° C. | 15 | 40.7 | 2.52 |
| 2 | 115° C. | 15 | 30.9 | 4.33 |

BZ is benzene and CHEX is cyclohexane.

### Example 9

This example of the invention evaluated a poly(amide)imide membrane for separation of a 25:25:25:25 admixture of benzene, heptane, cyclohexane and methylcyclopentane at a feed rate of 3 mL/hr. This poly(amide)imide membranes was made of TORLON® 4000T, which exhibited a Stability Rating of a level 3 pass, and had a nominal thickness of 1.5 mils. Runs for this example were carried out at oven temperatures of 115° C. and 130° C. and feed pressures of 14 psig. Observed results are summarized in Table XI.

TABLE XI

| | Separation | Separation | Separation | |
|---|---|---|---|---|
| | factor | factor | factor | Permeation Rate |
| TEMP | BZ/MCP | BZ/heptane | BZ/CHEX | (kg-mμ/m²-hr) |
| 115° C. | Benzene only | | | 0.30 |
| 130° C. | 16.9 | 7.8 | (no CHEX) | 0.41 |

BZ is benzene, MCP is methylcyclopentane, and CHEX is cyclohexane.

### Example 10

This example of the invention, the membrane evaluated in Example 9 was tested for separation of a 25:25:25:25 admixture of benzene, hexane, 1-hexene, and cyclohexane at a feed rate of 2.4 mL/hr. This example was carried out at an oven temperature of 100° C. and permeate side pressures of 14 psig. Observed results are summarized in Table XII.

TABLE XII

| Separation | Separation | Separation | |
|---|---|---|---|
| factor | factor | factor | Permeation Rate |
| BZ/hexane | BZ/hexene | BZ/CHEX | (kg-mμ/m²-hr) |
| 22.5 | 10.3 | 12.2 | 0.60 |

BZ is benzene, and CHEX is cyclohexane.

### Example 11

This example of the invention, the membrane evaluated in Example 9 was tested for separation for separation of a 20:20:20:20:20 admixture of benzene, hexane, 1-hexene, cyclohexane, and toluene at a feed rate of 2.4 mL/hr. This example was carried out at an oven temperature of 100° C. and feed pressures of 14 psig. Observed results are summarized in Table XIII, and a separation factor of 1.0 was observed for benzene/toluene.

TABLE XIII

| Separation | Separation | Separation | |
|---|---|---|---|
| factor | factor | factor | Permeation Rate |
| BZ/hexane | BZ/hexene | BZ/CHEX | (kg-mμ/m²-hr) |
| 14.6 | 7.4 | 10.1 | 2.91 |

BZ is benzene, and CHEX is cyclohexane.

That which is claimed is:

1. A process for recovery of one or more aromatic hydrocarbon compounds in admixture with other organic compounds which process comprises:
contacting a fluid mixture comprising two or more aromatic hydrocarbon compounds, with a first side of a perm-selective membrane comprising long-chain polymeric molecules in which recurring amide and imide linkages are part of the main polymer chain; and
selectively permeating at least one aromatic hydrocarbon compound of the mixture through the membrane to a permeate side that is opposite the first side, thereby exhibiting a separation factor of one aromatic hydrocarbon over another compound in a range upward from about 1.5.

2. The process of claim 1 wherein the polymeric membrane material is annealed at elevated temperatures in a range upward from about the glass transition temperature of the polymeric membrane material.

3. The process of claim 1 wherein the perm-selective membrane comprises a polymer derived from the reaction product of a carbocyclic aromatic primary diamine and an acyl halide derivative of trimellitic anhydride which contains at least one acyl halide group and that in the 4-ring position.

4. The process of claim 1 wherein the perm-selective membrane comprises a polymer derived from the reaction product of an aromatic diisocyanate and a tricarboxylic acid anhydride.

5. The process of claim 1 wherein the perm-selective membrane comprises a polymer derived from reactants selected from the group of reactant pairs consisting of (a) trimellitic anhydride and toluene diisocyanate, and (b) trimellitic anhydride chloride and toluene diamine.

6. The process of claim 1 wherein the perm-selective membrane comprises a polymer derived from the reaction product of trimellitic anhydride chloride and p-methylenediamine.

7. The process of claim 1 wherein the perm-selective membrane comprises a polymer derived from the reaction product of trimellitic anhydride chloride and a mixture of 4,4′-oxydianiline and m-phenylenediamine.

8. The process of claim 1 wherein the fluid mixture comprises para-xylene and least one other C₈ aromatic compound.

9. The process of claim 1 wherein the fluid mixture comprises the three isomers of xylene and optionally ethylbenzene, and the permeation exhibits a separation factor for para-xylene/meta-para-xylene of at least 1.5.

10. The process of claim 8 wherein the selective permeation is carried out at temperatures in a range downward from about 220° C. to about 70° C. and feed pressures up to 900 psia, and exhibits a para-xylene permeability of at least 0.5 Barrer.

11. A process for recovery of one or more aromatic hydrocarbon compounds in admixtures with other organic compounds which process comprises:
contacting a fluid admixture comprising hydrocarbon compounds having 4 or more carbon atoms, that includes at least one aromatic hydrocarbon compound, with a first side of a perm-selective membrane comprising long-chain polymeric molecules in which recurring amide and imide linkages are part of the main polymer chain, and the poly(amide)imide membrane material exhibited a Stability Rating of a level 3 pass; and
selectively permeating at least one aromatic hydrocarbon compound of the mixture through the membrane to a permeate side that is opposite the first side, thereby exhibiting a separation factor of one aromatic hydrocarbon over another compound in a range upward from about 2.5.

12. The process of claim 11 wherein the poly(amide)imide membrane material is annealed at elevated temperatures in a range upward from about the glass transition temperature of the polymeric membrane material.

13. The process of claim 11 wherein the fluid admixture comprises at least one aromatic hydrocarbon compound having 8 or more carbon atoms, and the compounds containing 4 or more carbon atoms are selected from the group consisting of alkenes, alkanes and alicyclic hydrocarbons.

14. The process of claim 11 wherein the perm-selective membrane comprises a polymer derived from reactants selected from the group of reactant pairs consisting of (a) trimellitic anhydride and toluene diisocyanate and (b) trimellitic anhydride chloride and toluene diamine.

15. The process of claim 11 wherein the perm-selective membrane comprises a polymer derived from the reaction product of trimellitic anhydride chloride and p-methylenediamine.

16. The process of claim 11 wherein the perm-selective membrane comprises a polymer derived from the reaction product of trimellitic anhydride chloride and a mixture of 4,4′-oxydianiline and m-phenylenediamine by a condensation polymerization.

17. A process for recovery of one or more aromatic hydrocarbon compounds in admixture with other organic compounds which process comprises:
contacting a gaseous mixture comprising two or more hydrocarbon compounds each having at least 5 carbon atoms, that includes at least one aromatic hydrocarbon compound, with a first side of a hollow fiber membrane comprising long-chain polymeric molecules in which recurring amide and imide linkages are part of the main polymer chain; and
selectively permeating at least one aromatic hydrocarbon compound of the mixture through the membrane to a permeate side that is opposite the first side first side, thereby exhibiting a separation factor of one aromatic hydrocarbon over another compound in a range upward from about 5.

18. The process of claim 17 wherein the poly(amide)imide membrane material exhibited a Stability Rating of a level 3 pass.

19. The process of claim 17 wherein the perm-selective membrane comprises a polymer derived from the reaction product of trimellitic anhydride chloride and a mixture of 4,4′-oxydianiline and m-phenylenediamine by a condensation polymerization.

20. The process of claim 17 wherein the poly(amide)imide membrane material is annealed at elevated temperatures in a range downward from about the glass transition temperature of the polymeric membrane material.

21. The process of claim 17 wherein the selective permeation is carried out under suitable operating conditions whereby the membranes exhibit a para-xylene permeability of at least 0.5 Barrer.

22. The process of claim 17 which further comprises recovering from the resulting mixture on the permeate side a permeate product enriched in one or more hydrocarbon compounds over that of the depleted mixture on the first side.

23. The process of claim 17 wherein the hollow fiber membrane comprises a polymer derived from trimellitic anhydride chloride and one or more carocyclic aromatic primary diamine, followed by final crosslinking after hollow fiber formation.

24. The process of claim 17 wherein the selective permeation is carried out at temperatures in a range downward from about 220° C. to about 70° C. and feed pressures up to 900 psia.

25. A process for recovery of one or more aromatic hydrocarbon compounds in admixture with other organic compounds which process comprises:
providing a perm-selective hollow fiber membrane, made by a method which comprises:
preparing an extrudable spinning solution comprising a poly(amide)imide polymer, which in the form of a membrane exhibits a Stability Rating of a level 3 pass, and a solvent system containing at least one organic compound;
extruding the spinning solution from an annular spinneret through an air gap into a quench bath containing water as a predominate component, while using a bore fluid, to from the hollow fiber; and
drying the hollow fiber;
contacting a fluid mixture comprising two or more aromatic hydrocarbon compounds, with a first side of a perm-selective, hollow fiber membrane comprising long-chain polymeric molecules in which recurring amide and imide linkages are part of the main polymer chain; and
selectively permeating at least one aromatic hydrocarbon compound of the mixture through the membrane to a permeate side that is opposite the first side, thereby exhibiting a separation factor of one aromatic hydrocarbon over another compound in a range upward from about 1.5.

26. The process of claim 25 wherein the solvent system comprises N-methyl-2-pyrrolidone.

27. The process of claim 27 wherein the bore fluid comprises N-methyl-2-pyrrolidone and water.

28. The process of claim 25 wherein the perm-selective hollow fiber membrane comprises a poly(amide)imide derived from the reaction product of trimellitic anhydride chloride and a mixture of 4,4′-oxydianiline and m-phenylenediamine by a condensation polymerization.

29. The process of claim 25 wherein the hollow fiber membrane is exposed in suitable conditions of temperature and pressure, for a time sufficient to enhance the perm-selective properties of the membrane.
